(19) United States
(12) Patent Application Publication
(10) Pub. No.: US 2019/0152047 A1
(43) Pub. Date: May 23, 2019

(54) BIOMECHANICAL ASSISTIVE DEVICE

(71) Applicant: STEERING SOLUTIONS IP HOLDING CORPORATION, Saginaw, MI (US)

(72) Inventors: Owen K. Tosh, Saginaw, MI (US); Zaki Ryne, Saginaw, MI (US); Muzaffer Y. Ozsecen, Saginaw, MI (US)

(21) Appl. No.: 16/044,712

(22) Filed: Jul. 25, 2018

Related U.S. Application Data
(60) Provisional application No. 62/588,645, filed on Nov. 20, 2017.

(51) Int. Cl. B25J 9/00; A61F 5/01; B25J 9/16

(52) U.S. Cl. CPC B25J 9/0006; A61F 5/0123; B25J 9/1628; A61B 5/4528; A61H 2201/018; A61H 2201/1207; A61F 2005/0155

(57) ABSTRACT

Technical solutions described herein include a biomechanical assistive device that includes a motor control system that generates assistive torque based on a torque profile that is associated with a present activity being performed by a user. The device further includes an activity module that detects a new activity being initiated by the user, and computes a confidence index of the new activity. In response to the confidence index being above a threshold, the activity module switches the biomechanical assistive device to use a torque that is associated with the new activity.

### CROSS-REFERENCES TO RELATED APPLICATIONS

This patent application claims priority to U.S. Provisional Patent Application Ser. No. 62/588,645, filed Nov. 20, 2017, which is incorporated herein by reference in its entirety.

### BACKGROUND

The present application generally relates to a biomechanical assistive device, and particularly to activity recognition by the assistive device.

Exoskeletons are devices that can amplify a person's natural ability and improve their quality of life. In one or more examples, exoskeleton devices facilitate overcoming physical human limitations by amplifying human strength, endurance, and mobility potential. The exoskeleton devices are thus biomechanical assistive devices that may be worn by a user, for example worn in association with a joint in the body, to amplify or improve the functioning of that joint.

Exoskeleton devices can be classified as either passive or powered devices. A passive device typically cannot generate and deliver energy external to the user, rather a passive device helps the user employ his own muscle power more effectively. Passive devices can include springs, and can store potential energy and deliver it in addition to the human motion. One example of exoskeleton-based passive assist is passive gravity support where the exoskeleton supports part of the user's weight. However, the exoskeleton cannot contribute to raise the user's center of gravity, for example when getting up from a chair.

A powered exoskeleton device on the other hand generates and supplies energy to the user through external means (i.e. electrical, hydraulic, etc), in one or more examples, in a continuous way, to help the user to elevate the center of mass of the body at one point or another by generating torque, for example using one or more actuators. The biomechanical assistive devices that are described herein are powered exoskeleton devices.

For operation of the assistive devices, the devices have to provide the appropriate amount of torque to assist with the user's activity, one way of providing such assist is done by detecting the user's current activity (ex. walking, standing, sitting). Typically, the assistive devices require direct user input, or are very slow to recognize activities automatically. Accordingly, there is a need for the assistive devices to automatically recognize user activity within a predetermined duration threshold.

### SUMMARY

Technical solutions are described for providing torque assist at a human joint, such as the hip joint, using a personal biomechanical assistive device. The technical solutions described herein facilitate the assistive device to automatically detect an activity that a user is engaged in and provide corresponding amount of assistive torque.

Technical solutions described herein include a biomechanical assistive device that includes a motor control system that generates assistive torque based on a torque profile that is associated with a present activity being performed by a user. The device further includes an activity module that detects a new activity being initiated by the user, and computes a confidence index of the new activity. In response to the confidence index being above a threshold, the activity module switches the biomechanical assistive device to use a torque that is associated with the new activity.

According to one or more embodiments, a method for generating assistive torque by a biomechanical assistive device includes generating a first torque command for generating the assistive torque using a motor control system of the assistive device according to a present activity being performed by a user that is wearing the biomechanical assistive device. The method further includes detecting a new activity based on one or more sensor signals, and computing a confidence index of the new activity being performed. The method further includes, in response to the confidence index of the new activity being greater than or equal to a predetermined threshold, generating a second torque command for generating the assistive torque according to the new activity.

According to one or more embodiments, a computer program product for determining a present state of a device includes computer readable storage medium with computer executable instructions therein. The computer executable instructions cause a processing circuit to perform receiving a plurality of confidence indices, each confidence index is associated with a respective state of the device has been detected. The processing circuit further performs arbitrating the received confidence indices to determine a new state, the arbitrating performed based on a present state and the new state, and transitioning the device to the new state.

These and other advantages and features will become more apparent from the following description taken in conjunction with the drawings.

### BRIEF DESCRIPTION OF THE DRAWINGS

FIG. 1 is a perspective view of an exemplary adjustable assist device 10 according to one or more embodiments;

FIG. 2 depicts an example controller according to one or more embodiments;

FIG. 3 depicts a block diagram for activity transition in the assistive device according to one or more embodiments;

FIG. 4A depicts an example transition table according to one or more embodiments;

FIG. 4B depicts an example transition between states according to one or more embodiments;

FIG. 4C depicts an example transition table for the assistive device according to one or more embodiments;

FIG. 5 depicts a block diagram of an exemplary arbitration module according to one or more embodiments;

FIG. 6 depicts a flowchart of determining the recognized activity according to one or more embodiments;

FIG. 7 depicts a block diagram of an activity recognition module according to one or more embodiments; and

FIG. 8 depicts an example activity recognition module for sitting according to one or more embodiments.

### DETAILED DESCRIPTION

An exoskeleton, particularly, an active exoskeleton is a biomechanical assistive device that provides torque assist at a human joint, such as the hip joint. A technical challenge exists for the assistive device to recognize the user's current activity (walking, standing, sitting, etc.) so that appropriate amount of assist torque can be generated and provided to the joint/user. Typically, the assistive devices either require a direct user input, such as buttons, to indicate the activity that is about to be performed, or is being performed.

The technical solutions described herein address such technical challenges by automatically detecting the activity being performed, or that is about to be started. A technical challenge with automatic recognition of the user activity is the time taken to identify the activity. If the activity recognition takes longer than a predetermined duration the user is not comfortable using the assistive device. Typical solutions to mitigate the time taken for the activity recognition is using more sensors, often in conjunction with user interaction. However, such solutions drive up the product cost as well as weight and complexity of the assistive device, and even reduce intuitiveness of using the assistive device in case direct input identifying an activity is received/requested from the user. Further, because of the delay in activity recognition, the assistive devices include devices to provide feedback to the user that the activity has been recognized, for example haptic feedback devices. Such constant user input complicates and may slow down the device, and waiting for the device to detect modes and provide feedback is slow and feels unnatural.

The technical solutions described herein address such technical challenges by facilitating the assistive device to intuitively recognize the user activity using a minimal number of sensors to recognize the current user activity and within a predetermined duration, such as 1 second. Thus, the user has no substantial waiting time for the activity recognition, and no feedback is required. Further, the technical solutions described herein reduce cost and weight of the assistive device. In addition, the user-experience of the assistive device is improved by making it intuitive and practically immediate.

The biomechanical assistive device typically operates as a (or using a) finite state machine. Typically, each activity may be considered a ‘state’ of the state machine and determining when to transition from one activity (state) to another is defined by the state machine. A finite state machine is broadly defined as a system with a finite number of discrete states, where each state has criteria to transition to one or more other states of the state machine.

A technical challenge for a state machine with numerous states and numerous transitions, is that the design and implementation of the state machine becomes difficult to maintain. For example, a state-flow diagram becomes difficult to understand, and it is further difficult to map the design elements to the implementation of the state machine. Further, if there is substantial redundancy in the transition criteria, optimizing the implementation can become difficult.

The technical solutions described herein address such technical challenges with state machine implementation and optimization. The technical solutions described herein use a confidence-based state machine that separates the (redundant) transition criteria processing from the transition arbitration logic. In one or more examples, the confidence-based state machine uses a transition table. Additional features and advantages of the technical solutions are described herein. It should be noted that the technical solutions of implementing and optimizing a state machine are described herein in the context of a biomechanical assistive device, however, the technical solutions are applicable in any other context where a finite state machine is used.

The technical solutions described herein use embodiments directed to a hip-joint assistive device, however it will be appreciated that the technical solutions can be implemented in assistive devices used at any other joint, limb, or extremity in a body such as the ankle, knee, or hip joint of a leg or the wrist, elbow, or shoulder joint of an arm. Also, the user can be a human or an animal. Additionally, for ease of explanation, the term “limb” may be used to describe a limb segment (such as a lower leg or an upper arm) attached to a joint of a limb.

It should be noted that although the technical solutions described herein use embodiments in the context of particular biomechanical assistive devices, the technical solutions can be used in other devices that use a state machine, such as in an electric power steering (EPS) systems for signal arbitration (position, torque, speed, etc.), in an EPS for loss of assist mitigation and arbitration. The technical solutions described herein can also be used in an automotive for collision avoidance for autonomous and semi-autonomous vehicles, or for calculating a safest path to pass a vehicle in front. Alternatively, or in addition, the technical solutions described herein are applicable in an EPS, such as a Steer by wire system for initialization process (checking clutch, hand wheel, road wheel sensors etc.), or other diagnostics to be performed. The above is a non-limiting, exemplary list of applications for the technical solutions herein.

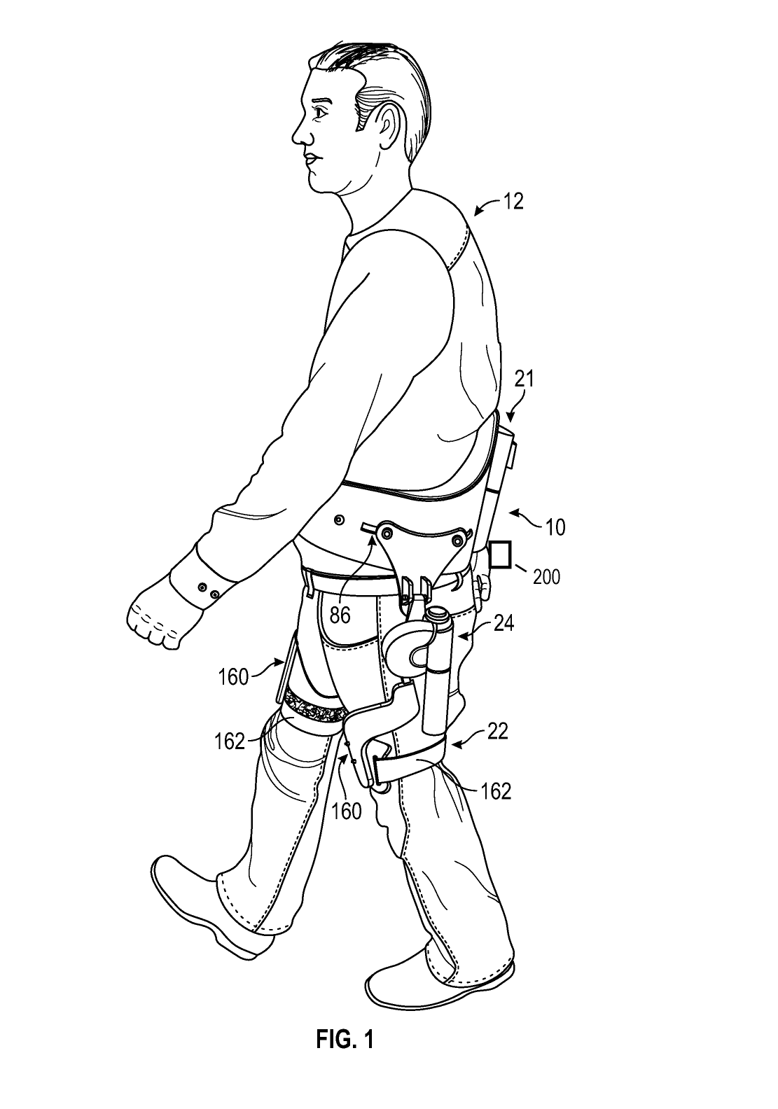

Referring to FIG. 1 an environmental view of a powered exoskeleton 10 that is attachable to a user 12 is shown. The powered exoskeleton 10 is wearable by the user 12 to aid the user 12 in performing various movements, tasks, or to reduce the user's energy consumption during various movements. The powered exoskeleton 10 is mechanically grounded to a portion of the user 12 to aid in the transfer of torque by the powered exoskeleton 10 to the user 12. The powered exoskeleton 10 includes a lumbar support apparatus 21, at least one leg support 22, and an actuator 24.

The lumbar support apparatus 21 is configured as a torso brace that interfaces with the user 12. The lumbar support apparatus 21 is disposed about a user's waist proximate a user's hip region. The lumbar support apparatus 21 is configured to adjust overall human-exoskeleton interface stiffness through the use of various lumbar support types. The various lumbar support types permit the user 12 to adjust for comfort and load or torque transfer efficiency from the powered exoskeleton 10 to the user 12. The device 10 further includes a controller 200. It should be noted that the depicted exoskeleton 10 is an example and that the technical solutions described herein are applicable to other types of biomechanical assistive devices too.

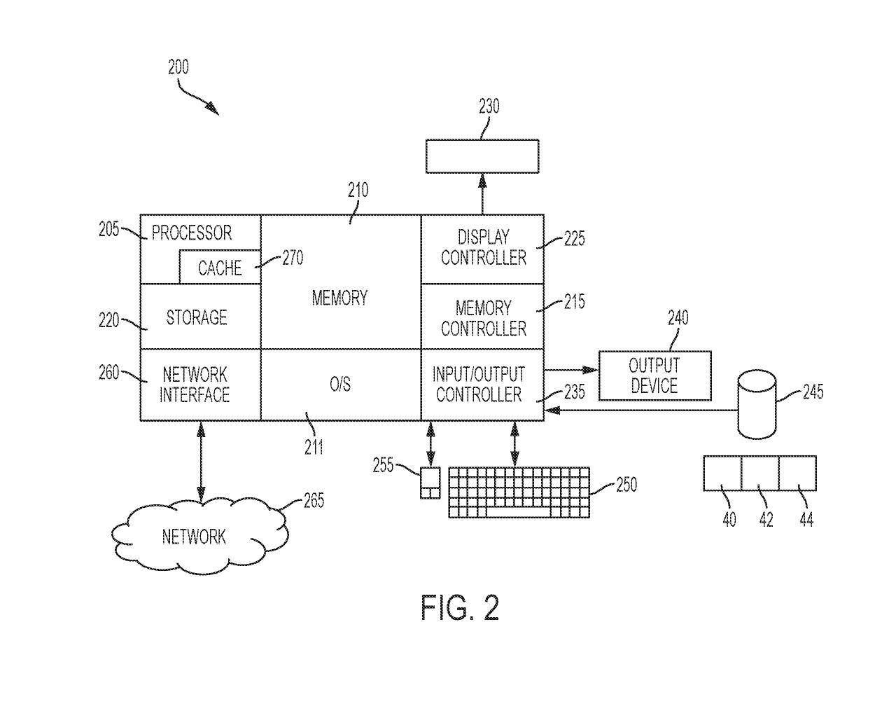

FIG. 2 depicts an example controller 200 according to one or more embodiments. The system 200 includes, among other components, a processor 205, memory 210 coupled to a memory controller 215, and one or more input devices 245 and/or output devices 240, such as peripheral or control devices, that are communicatively coupled via a local I/O controller 235. These devices 240 and 245 may include, for example, battery sensors, position sensors (gyroscope 40, accelerometer 42, GPS 44), indicator/identification lights and the like. Input devices such as a conventional keyboard 250 and mouse 255 may be coupled to the I/O controller 235. The I/O controller 235 may be, for example, one or more buses or other wired or wireless connections, as are known in the art. The I/O controller 235 may have additional elements, which are omitted for simplicity, such as controllers, buffers (caches), drivers, repeaters, and receivers, to enable communications.

The I/O devices 240, 245 may further include devices that communicate both inputs and outputs, for instance disk and tape storage, a network interface card (NIC) or modulator/demodulator (for accessing other files, devices, systems, or a network), a radio frequency (RF) or other transceiver, a telephonic interface, a bridge, a router, and the like.

The processor 205 is a hardware device for executing hardware instructions or software, particularly those stored in memory 210. The processor 205 may be a custom made or commercially available processor, a central processing unit (CPU), an auxiliary processor among several processors associated with the system 200, a semiconductor based microprocessor (in the form of a microchip or chip set), a macroprocessor, or other device for executing instructions. The processor 205 includes a cache 270, which may include, but is not limited to, an instruction cache to speed up executable instruction fetch, a data cache to speed up data fetch and store, and a translation lookaside buffer (TLB) used to speed up virtual-to-physical address translation for both executable instructions and data. The cache 270 may be organized as a hierarchy of more cache levels (L1, L2, and so on.).

The memory 210 may include one or combinations of volatile memory elements (for example, random access memory, RAM, such as DRAM, SRAM, SDRAM) and nonvolatile memory elements (for example, ROM, erasable programmable read only memory (EPROM), electronically erasable programmable read only memory (EEPROM), programmable read only memory (PROM), tape, compact disc read only memory (CD-ROM), disk, diskette, cartridge, cassette or the like). Moreover, the memory 210 may incorporate electronic, magnetic, optical, or other types of storage media. Note that the memory 210 may have a distributed architecture, where various components are situated remote from one another but may be accessed by the processor 205.

The instructions in memory 210 may include one or more separate programs, each of which comprises an ordered listing of executable instructions for implementing logical functions. In the example of FIG. 2, the instructions in the memory 210 include a suitable operating system (OS) 211. The operating system 211 essentially may control the execution of other computer programs and provides scheduling, input-output control, file and data management, memory management, and communication control and related services.

Additional data, including, for example, instructions for the processor 205 or other retrievable information, may be stored in storage 220, which may be a storage device such as a hard disk drive or solid state drive. The stored instructions in memory 210 or in storage 220 may include those enabling the processor to execute one or more aspects of the systems and methods described herein.

The system 200 may further include a display controller 225 coupled to a user interface or display 230. In some embodiments, the display 230 may be an LCD screen. In other embodiments, the display 230 may include a plurality of LED status lights. In some embodiments, the system 200 may further include a network interface 260 for coupling to a network 265. The network 265 may be an IP-based network for communication between the system 200 and an external server, client and the like via a broadband connection. In an embodiment, the network 265 may be a satellite network. The network 265 transmits and receives data between the system 200 and external systems. In some embodiments, the network 265 may be a managed IP network administered by a service provider. The network 265 may be implemented in a wireless fashion, for example, using wireless protocols and technologies, such as WiFi, WiMax, satellite, or any other. The network 265 may also be a packet-switched network such as a local area network, wide area network, metropolitan area network, the Internet, or other similar type of network environment. The network 265 may be a fixed wireless network, a wireless local area network (LAN), a wireless wide area network (WAN) a personal area network (PAN), a virtual private network (VPN), intranet or other suitable network system and may include equipment for receiving and transmitting signals.

In one or more examples, using only two position sensors (one for each hip position), the technical solutions described herein facilitates the assistive device to recognize a new activity of a user with no additional user input and transition to a torque profile for the new activity within the predetermined duration. For example, the assistive device identifies different activities of the user such as sitting, standing, sit-to-stand, stand-to-sit, and walking, and other such activities, and facilitates near real-time transition from one activity (present activity) to another activity (new activity) that the user began without any explicit input from the user identifying the new activity. The technical solutions described herein thus facilitate an intuitive operation of the assistive device for the user, in turn improving the performance of the assistive device.

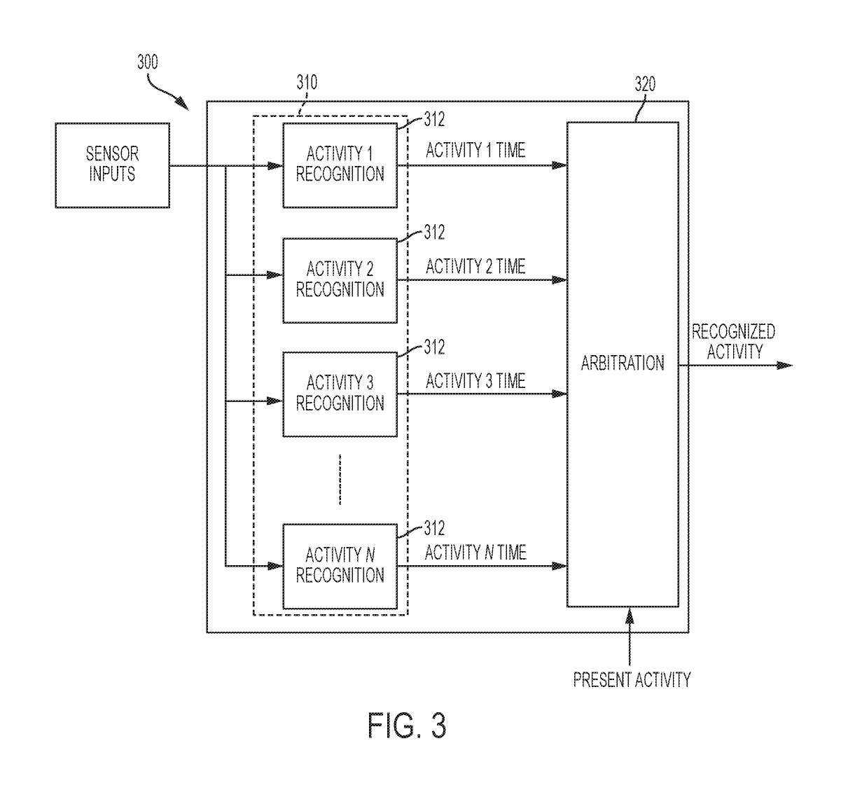

FIG. 3 depicts a block diagram for activity transition in the assistive device according to one or more embodiments. In one or more examples, the activity transition is performed by an activity module 300 that is part of the controller 200. As depicted in FIG. 3, the activity module 300 includes an activity recognition module 310 that includes one or more separate activity recognition modules 312 for specific activities that the assistive device 10 can help the user perform.

Each activity has separate criteria to be recognized, some of which may overlap. This includes thresholds and ranges for leg position and velocity, and their relationship to each other. For example, Sitting is detected when both legs are together and neither leg is moving, and Walking is detected when legs move in opposite directions. All activity criteria are evaluated in parallel; when an activity is recognized, a corresponding timer for that activity begins counting up. When the activity is no longer recognized, the corresponding timer resets. The values of these timers are passed to an arbitration unit 320 to determine the recognized activity as a final state. The recognized activity may be the new activity that the user is about to begin or the present activity that the user continues to perform.

The arbitration unit 320 determines the recognized activity based on the present activity and the time for each activity that the respective activity recognition modules 312 provide. Each potential transition has a timing threshold; that is, once an activity-recognition timer of an activity recognition module 312 passes a unique threshold corresponding to the present activity, the arbitration unit 320 outputs the activity corresponding to the activity recognition module 312 as the recognized activity. This results in a two-dimensional transition table implementation, which can expand as other activities are added.

The assistive device 10 associates a “confidence index” to each state (i.e. activity) that is part of the finite state machine in which the assistive device 10 operates. The confidence index of a state represents an entry criteria for the particular state, each state having its respective confidence index. In one or more examples, the confidence index can be realized with one or more parameters (e.g. time). In one or more examples, all confidence indices in the assistive device 10 have the same ‘meaning’ relative to each other although they can each be calculated in completely different ways. Typically, a confidence index increases as the criteria are more “fully” met, or over time as the criteria are continuously met.

The confidence indices are calculated independently from one another. This means that there can be overlap between the criteria (i.e. two Confidence Indices may increase in parallel), and changes in the criteria for one state may or may not impact any other state. In one or more examples, the arbitration module 320 uses only the confidence indices and the current state of the state machine (i.e. assistive device 10) for its operation to determine a state transition. The configuration of the arbitration module 320, in one or more examples, is represented in a transition table.

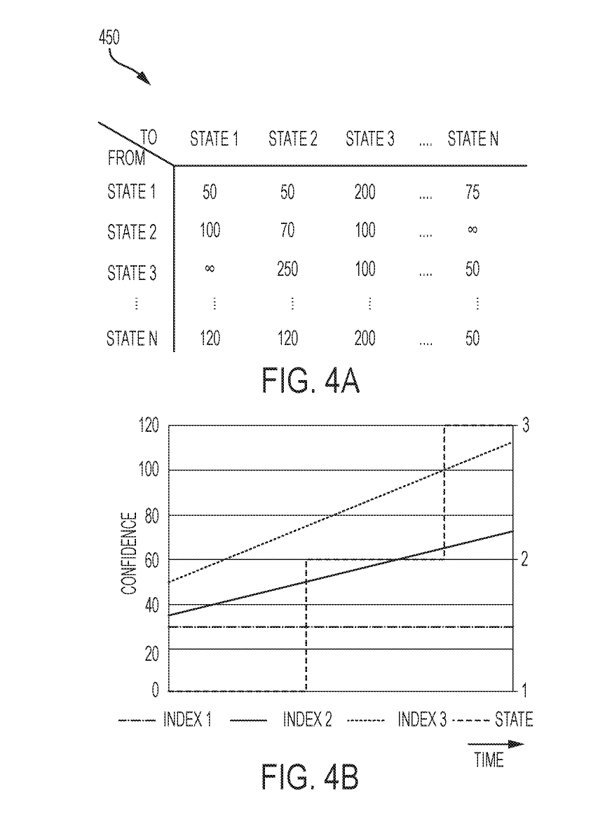

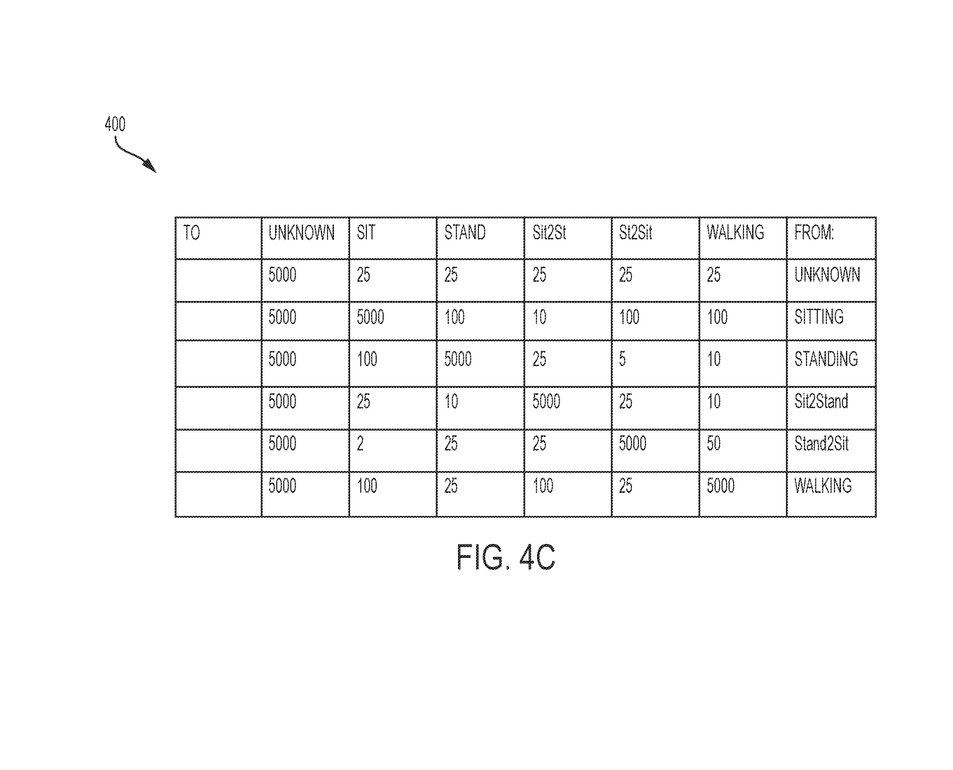

FIG. 4A depicts an example transition table according to one or more embodiments. As depicted, the transition table 450 includes the confidence index thresholds for a state transition from one state to another. Each row-column pair represents a confidence index threshold for a transition from a first state (e.g. row) to a second state (e.g. column). The present state of the state machine (i.e. the assistive device 10) is used to select which row of the table 450 is used. Then, comparisons are performed for all computed confidence indices against each of the thresholds in the present state's row. If the confidence index threshold at a column Y meets or exceeds the computed confidence index for the state indicated in column Y, a state transition is performed. In the case of two or more indices simultaneously meeting (or exceeding) the table thresholds, the implementation can define a priority scheme (fixed priorities, prioritize current state, etc.) to determine which state to transition into. It should be noted that the threshold values used are calibrateable. In one or more examples, the threshold values are adjusted dynamically or calibrated when adjusting the assistive device for a particular user.

The technical solutions described herein, accordingly, split the implementation of the state machine of the assistive device 10 into two stages—a first stage where entry criteria for each state is defined, resulting in a plurality of confidence indices; and a second stage where an arbitration determines a next state (activity) that a user wants to transition into based on the previously detected state, and the continuously monitored confidence indices. In an example, the threshold confidence indices that are stored in the transition table 450 are dynamically updated.

As described herein, the arbitration module 320 accesses the values corresponding to a present state that the assistive device 10 is in and determines the next state to transition into based on the confidence index thresholds in the transition table 450. If a specific row-column pair does not have a valid confidence index threshold value, the assistive device 10 cannot transition from the first activity (row) to the second activity (column) corresponding to the pair. For example, an invalid entry may be an ‘infinity’ value stored in the entry in the transition table 450. Else, if a valid confidence index threshold value is stored in the entry, and once the state's confidence index meets or exceeds the table value, a state transition occurs.

FIG. 4B depicts an example transition between states according to one or more embodiments. In the example shown, the state machine (assistive device 10) starts in a state-1. In this example, the confidence index for state-1 is constant, while the indices for states-2 and -3 increase at different rates over time. The first state transition occurs when the confidence index for state-2 reaches a value of 50, which meets the threshold value in table 450 (first row, second column). The second state transition occurs when the confidence index for state 3 reaches 100, which meets the threshold value in table 450 (second row, third column).

FIG. 4C depicts an example transition table for the assistive device according to one or more embodiments. The transition table 400 represents combinations of activity-pairs, each activity-pair being a transition from a first activity to a second activity in the pair. For example, consider that the transition table 400 includes possible present activities along the Y axis and the possible recognized activities along the X axis. In this case, each entry (i, j) in the table 400 represents transition from an activity-i to an activity-j. The entry (i, j) specifies the unique threshold for the transition from the activity-i to the activity-j. For example, referring to FIG. 4C, a threshold to determine a transition from Standing (activity-i; i=3) to Stand-to-Sit (activity-j; j=5) is 5 ms, while a threshold to determine a transition from Standing (activity-i; i=3) to Walking (activity-j; j=6) is 10 ms. Similarly, the transition table 400 includes other thresholds corresponding to the other possible combinations of present activities and recognized activity. It should be understood that the threshold values depicted in FIG. 4C are exemplary and that in other examples different threshold values can be used for the depicted activity pairs. Further, it should be noted that the activities listed in FIG. 4C are exemplary and that in other examples, different and/or additional activities may be used.

The arbitration unit thus detects transitions (e.g. Standing to Walking), which are more probable, faster than less probable transitions (e.g. Sitting to Walking). By allowing the assistive device 10 to detect the less probable transitions increases the robustness of the assistive device, as any incorrectly detected states do not result in a permanent error condition.

Accordingly, the technical solutions described herein facilitate activity recognition using a minimal number of sensors, which can be expanded with further activities and transitions. The arbitration unit 320 facilitates fast, intuitive activity switching (e.g. less than 250 milliseconds for transitions).

The recognized activity is used to determine a torque command to be provided to the motor system 14. Alternatively, or in addition, the recognized activity is used to determine a motor velocity command to be provided to the motor system 14. The motor system 14 uses the input commands to operate the one or more motors of the assistive device 10 to generate a corresponding amount of torque and/or displacement of the motor to provide the assist to the user.

In one or more examples, the assistive device 10 uses a torque profile particular to the activity being performed by the user. For example, the assistive device 10 uses a walking torque profile to generate the assistive torque when the user is performing ‘walking’, a sitting torque profile when the user is performing ‘sitting’ and so on. Each torque profile may include one or more gain factors that are specific to the activity to generate different assistive torque for the specific activities. In such cases, the controller 200 selects the specific torque profile for generating the torque command/velocity command for the motor system 14 based on the recognized activity determined by the activity module 300. If the present activity is the same as the recognized activity the existing torque profile in use is continued, else the controller 200 switches the torque profile from that of the present activity to the torque profile of the recognized activity.

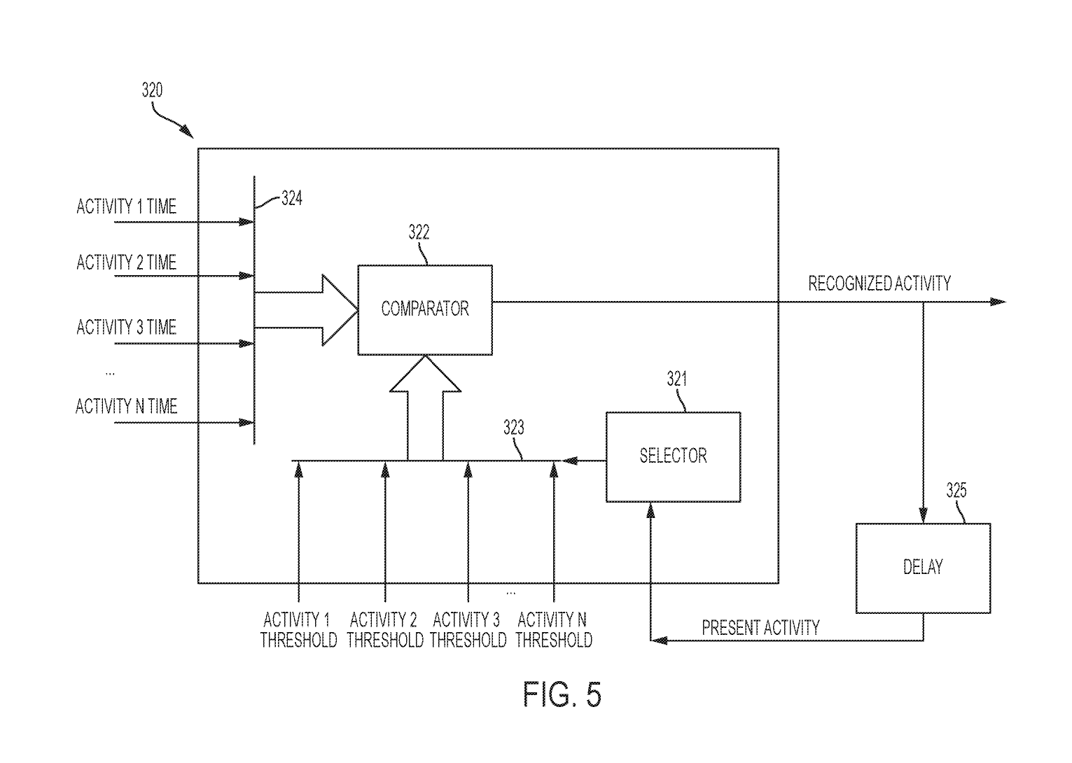

FIG. 5 depicts a block diagram of an exemplary arbitration module according to one or more embodiments. The arbitration module 320 depicted includes a selector module 321 and a comparator module 322, among other modules.

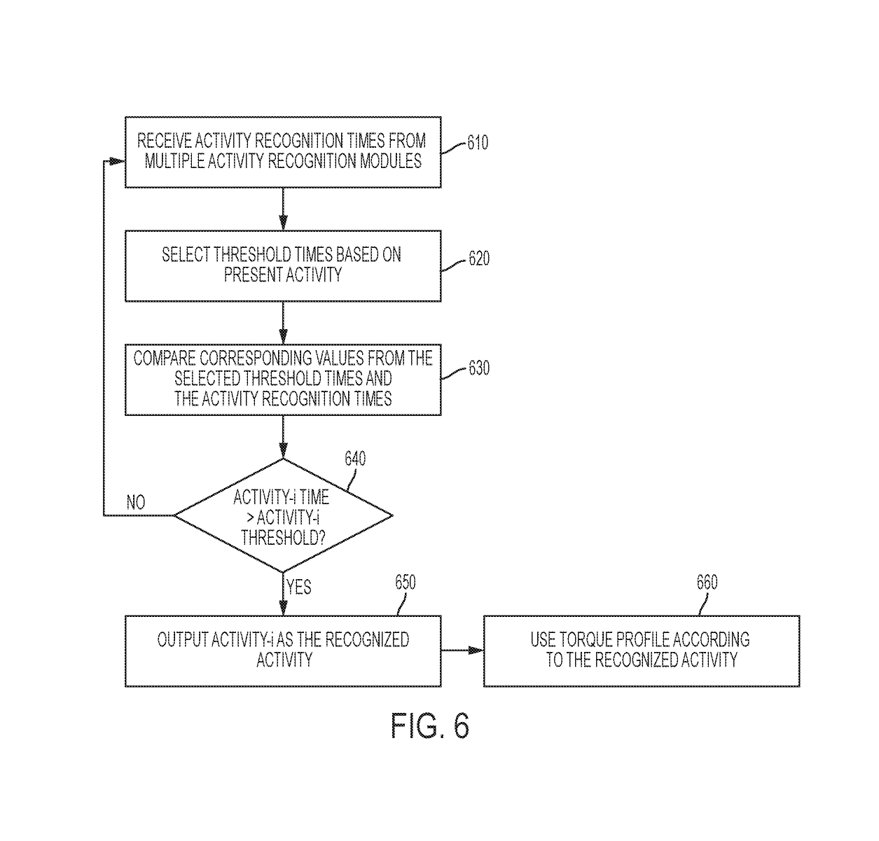

FIG. 6 depicts a flowchart of determining the recognized activity according to one or more embodiments. The method is executed by the arbitration module 320. The method is described with reference to FIG. 5 and FIG. 6. The arbitration module 320 receives the timer results from each of the activity recognition modules 312, which are received by the comparator 322 as input 324 (block 610). In one or more examples, the activity times received are passed through a saturation and limitation module (not shown) prior to being received by the comparator 322. The input 324 includes multiple activity recognition times, for example, an activity recognition time from each activity recognition module 312 that detects a corresponding activity based on the sensor inputs. In one or more examples, the input 324 includes an activity recognition time for each possible activity. Alternatively, or in addition, the input 324 includes an activity recognition time only for a subset of the possible activities, for example for Sitting, SitToStand, and StandToSit. In one or more examples, if an activity is not being detected, the corresponding activity recognition module 312 sends a predetermined value, for example 0, indicating that that activity is not being detected for any recent time.

The comparator module 322 further receives as input 323 a subset of threshold times from the transition table, for example, a row of the transition table 400, based on the present activity for which the assistive device 10 is assisting. The input 323 includes the predetermined threshold times for determining a new activity based on the present activity. The selector module 321 identifies and selects the subset of threshold times as the input 323 to compare with the input 324 based on the present activity (block 620). In one or more examples, the present activity is input to the selector 322 as a delayed output of the arbitration module 320 itself using a delay unit 325. In one or more examples, the selected input 323 is one row of threshold times from the transition table 400, the row corresponding to the present activity. For example, referring to the exemplary transition table 400 depicted in FIG. 4B, if the present activity is ‘standing’ the input 323 includes the row <65535, 100, 65535, 250, 50, 100> corresponding to the ‘standing’ activity.

The comparator 322 compares the corresponding values from the input 323 and the input 324 with each other to determine the recognized activity (block 630). For example, the comparator 322 compares the activity-i time in the input 324 (from the activity recognition modules) with the activity-i threshold in the input 323 (from the transition table 400).

If the comparator 322 detects that the activity-i time has passed the activity threshold time corresponding to the present activity, the comparator indicates the activity-i as the recognized activity (blocks 640 and 650). For example, consider the example where the present activity is standing. In this case the activity recognition times are compared with the threshold times provided in the corresponding row in the transition table 400. If the activity recognition time for the activity ‘standing to sitting’ has crossed 50 ms, the arbitration unit 320 outputs ‘standing to sitting’ as the recognized activity, and the assistance device 10 generates torque command/switches torque profile accordingly. Alternatively, if the ‘standing to sitting’ has not yet crossed the threshold, and instead if the ‘sitting’ activity has been detected for at least the last 1000 ms (more than corresponding threshold), the arbitration unit 320 outputs ‘sitting’ as the recognized activity.

The assistive device 10 selects the torque profile according to the output recognized activity (block 660). Using the torque profile can include generating one or more torque/motor commands for the motor control system 14 to generate the assistive torque corresponding to the recognized activity. If the recognized activity is different than the present activity, the controller 200 switches the torque profile from that of the present activity to the recognized activity.

The method is executed continuously in a loop so that a new activity that the user is about to initiate can be intuitively recognized by the assistive device 10, without any direct input from the user to indicate what activity is being started. Thus, the method facilitates the assistive device 10 to recognize a new activity that the user is about to begin from the present activity that the user is already performing. The recognition is performed with minimal delay (less than 250 milliseconds) and using minimal sensors (2 position sensors), driving the cost and weight of the assistive device lower than devices using additional sensors.

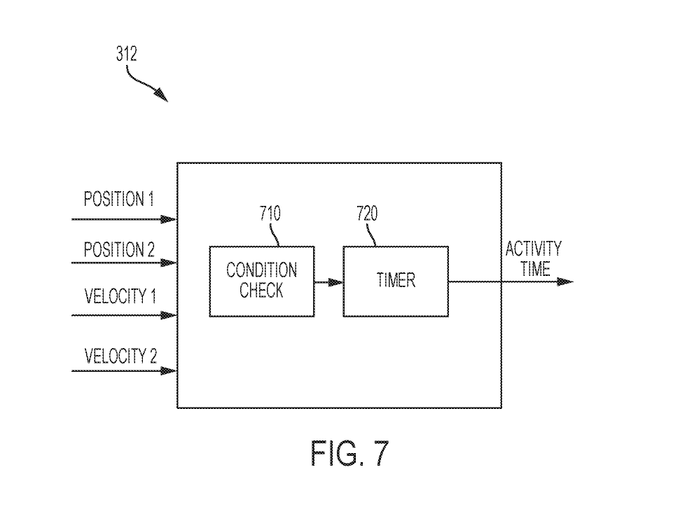

FIG. 7 depicts a block diagram of an activity recognition module according to one or more embodiments. An activity recognition module 312 includes a condition check module 710 and a timer 720, among other components. The condition check module is configured to receive inputs from one or more sensors. In the present example of using minimal sensors, particularly only two position sensors, the condition check module 710 receives signals from the two position sensors of the assistive device 10. The position sensors in case of the hip joint assistive device can be placed on the two sides, one for each hip of the user. The input signals received by the condition check module 710 can include a first position signal and a first velocity signal from the first position sensor, and a second position signal and a second velocity signal from the second position sensor. It should be noted that in some cases, the activity recognition module 312 may receive fewer sensor signals.

The condition check module 710 determines if a predetermined condition for a specific activity is met and in such a case, activates the timer 720. The timer measures a duration for which the condition check module 710 determines that the predetermined condition is being met by the one or more input signals. If the condition stops being true, the condition check module 710 disables the timer 720. In one or more examples, disabling the timer 720 can also reset the timer 720. The timer 720 thus indicates an amount of time for which the activity was recognized according to the condition check module 710. The condition checked is different for different activities.

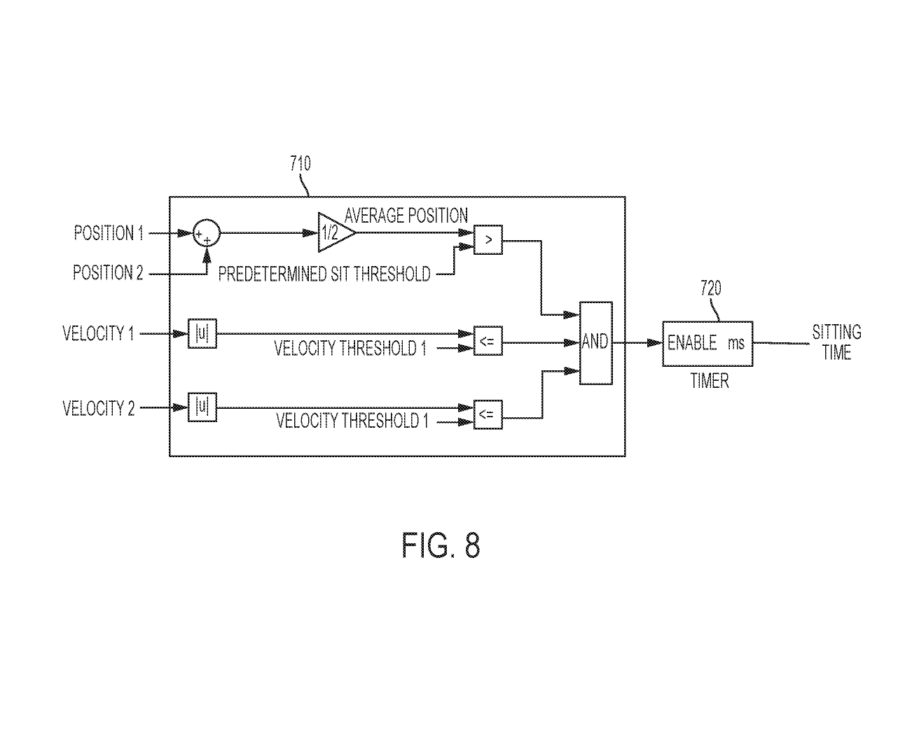

FIG. 8 depicts an example activity recognition module for sitting according to one or more embodiments. In the depicted activity recognition module for sitting, the condition check module 710 computes an average position using the two position signals. Further, the average position is compared with a predetermined position threshold. In addition, the condition check module 710 compares each of the velocity signals with a predetermined velocity threshold. If the average position is greater than the predetermined position threshold, and each velocity is less than the predetermined velocity threshold, the timer 720 is enabled. The output of the timer 720 represents an amount of time sitting was detected. Similarly, different criteria can be used for other activity recognitions, such as for standing, sit-to-stand, stand-to-sit, walking, and the like.

The activity recognition times from the one or more different activity recognition modules are further used for detecting the transition from one activity to another without any additional input from the user and without any additional sensors than the two position sensors used for detecting the activities. Based on the transition, the assistive device 10 can switch a torque profile and/or generate a torque command for generating assistive torque for the new activity that the user is performing. The transition is within a predetermined threshold intuitive as the user simply can move from one activity to another, without communication with the assistive device or waiting for the assistive device to switch from one mode to another. The technical solutions described herein thus improve the performance of an assistive device.

Further yet, the technical solutions use additional sensors and user input devices to capture additional measurements. In one or more examples, the technical solutions described herein facilitate the assistive device to capture user specific information, such as the user height, weight, and other body measurements in addition to sensor measurements. In one or more examples, the assistive device captures parameter measurements and data specific to one or more clinical functions. The data captured corresponding to the one or more clinical functions is further provided for further analysis and reporting via one or more communication channels. In one or more examples, the data may be provided to an external analysis system.

While the technical solutions has been described in detail in connection with only a limited number of embodiments, it should be readily understood that the technical solutions are not limited to such disclosed embodiments. Rather, the technical solutions can be modified to incorporate any number of variations, alterations, substitutions or equivalent arrangements not heretofore described, but which are commensurate with the spirit and scope of the technical solutions. Additionally, while various embodiments of the technical solutions have been described, it is to be understood that aspects of the technical solutions may include only some of the described embodiments. Accordingly, the technical solutions are not to be seen as limited by the foregoing description.

What is claimed is:

1. A biomechanical assistive device comprising:
a motor control system configured to generate assistive torque based on a torque profile that is associated with a present activity being performed by a user;
an activity module configured to:
detect a new activity being initiated by the user;
compute a confidence index of the new activity; and
in response to the confidence index being above a threshold, switch the biomechanical assistive device to use a torque that is associated with the new activity.

2. The biomechanical assistive device of claim 1, wherein the threshold is specific to the present activity being performed by the user.

3. The biomechanical assistive device of claim 2, wherein the threshold is further specific to the new activity that is detected.

4. The biomechanical assistive device of claim 1 further comprising:
an activity recognition module configured to determine an activity being performed by the user automatically based on one or more sensor signals.

5. The biomechanical assistive device of claim 4 wherein, the sensor signals are received from a hip-joint of the user.

6. The biomechanical assistive device of claim 5, the sensor signals being kinematics signals comprising a pair of position signals and a pair of velocity signals.

7. The biomechanical assistive device of claim 1, wherein the activity module switches from the present activity to the new activity without a dedicated user input indicating change in an activity.

8. The biomechanical assistive device of claim 1, the activity module comprising a transition table comprising a plurality of predetermined thresholds corresponding to pairs of the present activity and the new activity.

9. The biomechanical assistive device of claim 1, wherein the confidence index is computed based on an amount of time the new activity is performed.

10. A method for generating assistive torque by a biomechanical assistive device, the method comprising:
generating a first torque command for generating the assistive torque using a motor control system of the assistive device according to a present activity being performed by a user that is wearing the biomechanical assistive device;
detecting a new activity based on one or more sensor signals;
computing a confidence index of the new activity being performed; and
in response to the confidence index of the new activity being greater than or equal to a predetermined threshold, generating a second torque command for generating the assistive torque according to the new activity.

11. The method of claim 10, further comprising:
automatically switching to the new activity being performed by the user that is wearing the biomechanical assistive device based on the sensor signals.

12. The method of claim 10, wherein the predetermined threshold is specific to the present activity being performed by the user.

13. The method of claim 12, wherein the predetermined threshold is further specific to the new activity that is detected.

14. The method of claim 10, further comprising:
determining the predetermined threshold from a transition table that comprises a plurality of predetermined thresholds corresponding to pairs of activities, the predetermined threshold determined based on the present activity and the new activity.

15. A computer program product for determining a present state of a device, the computer program product comprising computer readable storage medium with computer executable instructions therein, the computer executable instructions cause a processing circuit to perform:
receiving a plurality of confidence indices, each confidence index is associated with a respective state of the device has been detected;
arbitrating the received confidence indices to determine a new state, the arbitrating performed based on a present state and the new state; and
transitioning the device to the new state.

16. The computer program product of claim 15, wherein the each of the confidence indices are received from respective state detection modules, each activity recognition module detecting a corresponding state.

17. The computer program product of claim 16, wherein each of the state detection modules detects the corresponding state based on one or more criteria.

18. The computer program product of claim 15, wherein the arbitration is performed based on a predetermined threshold that is specific to the present state of the device.

19. The computer program product of claim 18, wherein the predetermined threshold is further specific to the new state.

20. The computer program product of claim 18, the computer executable instructions cause the processing circuit to perform:
determining the predetermined threshold from a transition table that comprises a plurality of predetermined thresholds corresponding to pairs of states, the predetermined threshold determined based on the present state and the new state.

21. The computer program product of claim 15, wherein the device generates an amount of torque based on the new state.
